(19) United States
(12) Patent Application Publication
(10) Pub. No.: US 2021/0365868 A1
(43) Pub. Date: Nov. 25, 2021

(54) SYSTEMS AND METHODS FOR ALLOCATING AGENT RESOURCES IN A CONTACT CENTER

(71) Applicant: Avaya Inc., Santa Clara, CA (US)

(72) Inventor: Thomas Moran, Galway City (IE)

(21) Appl. No.: 17/444,244

(22) Filed: Aug. 2, 2021

Related U.S. Application Data
(63) Continuation of application No. 15/086,205, filed on Mar. 31, 2016, now abandoned.

(51) Int. Cl. G06Q 10/06; B64C 39/02; G08G 5/00

(52) U.S. Cl. CPC G06Q 10/063112; B64C 39/024; G08G 5/0069; G16H 40/67; B64C 2201/145; B64C 2201/146; G08G 5/0026

(57) ABSTRACT

Allocating agent resources in a contact center including receiving a new contact at the contact center and instantiating a contact object corresponding to the new contact. A first set of information is received by the contact center, and a first unmanned aerial vehicle (UAV) is selected for deployment to a target destination. At least one characteristic of the first UAV is associated with the contact object; an additional set of information related to at least one of the contact object or the first UAV is received by the contact center during a travel period of the first UAV to the target destination; and a dynamic prediction is made regarding a particular agent of the contact center to connect to the contact object. The contact object is then connected to an agent device associated with the particular agent.

### CROSS-REFERENCE TO RELATED APPLICATIONS

This application is a continuation of U.S. patent application Ser. No. 15/086,205 filed on Mar. 31, 2016, entitled “SYSTEMS AND METHODS FOR ALLOCATING AGENT RESOURCES IN A CONTACT CENTER,” the entirety of which is hereby incorporated by reference.

### BACKGROUND

The present disclosure relates generally to operation of a contact center, and, more particularly, to use of unmanned aerial vehicles (UAVs) with the contact center.

The use of UAVs or drones is a rapidly evolving technology area with the potential for continued growth in many commercial and public health and safety applications such as medical emergency services, surveillance, media coverage, goods delivery, etc. Skilled human personnel are a scarce resource and represent a large percentage of the operating cost in many businesses. Because UAVs are typically fully or partially autonomous, they may operate at a much lower cost than human personnel. However, UAVs typically still require input from a human agent at one or more time points during the UAV excursion such as during takeoff and landing and when the UAV reaches its destination.

### BRIEF SUMMARY

One aspect of the present disclosure relates to a method for allocating agent resources in a contact center. This method includes receiving a new contact at the contact center, and instantiating, by the processor of the contact center, a contact object corresponding to the new contact, and receiving, by the processor of the contact center, a first set of information related to the contact object. This method also includes selecting, by the processor of the contact center, a first UAV from a plurality of UAVs for deployment to a target destination, in which the first UAV is selected based on the first set of information and at least one feature of each of the plurality of UAVs; and associating, by the processor of the contact center, at least one characteristic of the first UAV with the contact object. This method also includes receiving, by the processor of the contact center, during a travel period of the first UAV to the target destination, an additional set of information related to at least one of the contact object or the first UAV; dynamically predicting, by the processor of the contact center, a particular agent of the contact center to connect to the contact object, in which the prediction is based on the first set of information and the additional set of information; and connecting, by the processor of the contact center, the contact object to an agent device associated with the particular agent of the contact center.

Another aspect of the present disclosure relates to a system for allocating agent resources in a contact center. This system includes a memory device, storing executable instructions, and a processor in communication with the memory device. In particular, the processor when executing the executable instructions: upon receipt of a new contact, instantiates a contact object corresponding to the new contact, in which the new contact comprises a request for assistance for a user at a target destination; receives a first set of information related to the contact object; selects a first UAV from a plurality of UAVs for dispatch to the target destination, in which the first UAV is selected based on the first set of information and at least one feature of each of the plurality of UAVs; associates at least one characteristic of the UAV with the contact object; receives an additional set of information related to at least one of the contact object or the first UAV, in which the additional set of information is received during a travel period of the first UAV to the target destination; dynamically predicts a particular agent of the contact center to connect to the contact object, in which the prediction is based on the first set of information and the additional set of information; and connects the contact object to an agent device associated with the particular agent of the contact center.

A further aspect of the present disclosure relates to a method for operating a UAV under direction from a contact center. This method includes receiving, by a processor of the UAV, dispatch instructions from a computer system of the contact center; in which a) the dispatch instructions comprise a target destination associated with a new contact received by the contact center, the new contact comprising a request from a user; and b) the UAV is selected by the computer system of the contact center from a plurality of available UAVs for dispatch based on a first set of information received by the contact center related to the contact object and at least one feature of each of the plurality of UAVs. This method also includes controlling, by the processor of the UAV, operation of the UAV to navigate the UAV to the target destination; transmitting, by the processor of the UAV, to the contact center, an additional set of information related to the UAV, in which the additional set of information is transmitted during a travel period of the UAV to the target destination; and receiving, by the processor of the UAV, from the computer system of the contact center, communication instructions. This method also includes, in response to receiving the communication instructions, controlling, by the processor of the UAV, an active communication link between the user and a particular agent of the contact center, in which the particular agent is selected by the computer system of the contact center based on the first set of information and the additional set of information.

### BRIEF DESCRIPTION OF THE DRAWINGS

So the manner in which the above recited features of the present disclosure may be understood in detail, a more particular description of embodiments of the present disclosure, briefly summarized above, may be had by reference to embodiments, which are illustrated in the appended drawings. It is to be noted, however, the appended drawings illustrate only typical embodiments encompassed within the scope of the present disclosure, and, therefore, are not to be considered limiting, for the present disclosure may admit to other equally effective embodiments, wherein:

FIG. 1 illustrates a contact center architecture in accordance with the principles of the present disclosure;

FIG. 2 illustrates a server in accordance with principles of the present disclosure;

FIG. 3 illustrates an example communication architecture between a user, an agent of the contact center, and one or more UAVs in accordance with principles of the present disclosure;

FIGS. 4 and 5 are flowcharts of exemplary methods for allocating agent resources in accordance with principles of the present disclosure; and

FIG. 6 is a top view of a simplified illustration depicting a UAV in accordance with principles of the present disclosure.

### DETAILED DESCRIPTION

The present disclosure will be illustrated below in conjunction with an exemplary communication system. Although well suited for use with, e.g., a system having an Automated Call or Contact Distribution (ACD) system or other similar contact processing switch, the present disclosure is not limited to any particular type of communication system switch or configuration of system elements. Those skilled in the art will recognize that the disclosed techniques may be used in any communication application in which it is desirable to provide improved contact processing.

In the following detailed description, numerous specific details are set forth in order to provide a thorough understanding of embodiments or other examples described herein. In some instances, well-known methods, procedures, components and circuits have not been described in detail, so as to not obscure the following description. Further, the examples disclosed are for illustrative purposes only and should not be construed as limiting of the scope of embodiments of the present disclosure.

The term “a” or “an” entity refers to one or more of that entity. As such, the terms “a” (or “an”), “one or more” and “at least one” may be used interchangeably herein. It is also to be noted that the terms “comprising”, “including”, and “having” may be used interchangeably.

The term “automatic” and variations thereof, as used herein, refers to any process or operation done without material human input when the process or operation is performed. However, a process or operation may be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material.”

As used herein in connection with embodiments of the present disclosure, the term “contact” refers to a communication from a user or customer. The communication may be by way of any communication medium such as, but not limited to, a telephone call, e-mail, instant message, web chat, and the like. The terms “user” and “customer” denote a party external to the contact center and are used herein to refer to the other party to a contact or a communications session. A user or customer may include, for example, a person requesting assistance for himself or herself or a person requesting assistance for a third party and may also include, for example, a person having a commercial relationship with the contact center or with a business represented by the contact center. In some embodiments, the “user” may include, for example, an automated monitoring or alarm system that utilizes various sensors to detect, for example, a potential unauthorized entry, and sends a signal to the contact center.

For each “contact” received by the contact center, a computer of the contact center will instantiate a “contact object,” which may comprise one or more variables, functions, and data structures that represent the contact. Each contact object comprises a number of attributes, which are assigned values based on its corresponding contact. Each contact object can also include pointers, links or references to other data and/or attributes associated with its corresponding contact. The contact object may be passed amongst the different queues, databases, systems, software applications and other computer-based resources of the contact center. Each contact object may also include a forward reference to another contact object and a backward reference to yet another contact object. In this manner, a prioritized list, or queue, of contact objects may be constructed.

As used herein, the term “transmitter” may generally comprise any device, circuit, or apparatus capable of transmitting a signal. As used herein, the term “receiver” may generally comprise any device, circuit, or apparatus capable of receiving a signal. As used herein, the term “transceiver” may generally comprise any device, circuit, or apparatus capable of transmitting and receiving a signal. As used herein, the term “signal” may include one or more of an electrical signal, a radio signal, an optical signal, an acoustic signal, and so forth.

The term “computer-readable medium” as used herein refers to any tangible storage and/or transmission medium that participates in storing and/or providing instructions to a processor for execution. Such a medium may take many forms, including but not limited to, non-volatile media, volatile media, and transmission media. Non-volatile media includes, for example, NVRAM, or magnetic or optical disks. Volatile media includes dynamic memory, such as main memory. Common forms of computer-readable media include, for example, a floppy disk, a flexible disk, hard disk, magnetic tape, or any other magnetic medium, magneto-optical medium, a CD-ROM, any other optical medium, punch cards, paper tape, any other physical medium with patterns of holes, RAM, PROM, EPROM, FLASH-EPROM, solid state medium like a memory card, any other memory chip or cartridge, a carrier wave as described hereinafter, or any other medium from which a computer may read. A digital file attachment to e-mail or other self-contained information archive or set of archives is considered a distribution medium equivalent to a tangible storage medium. When the computer-readable media is configured as a database, it is to be understood that the database may be any type of database, such as relational, hierarchical, object-oriented, and/or the like. Accordingly, the disclosure is considered to include a tangible storage medium or distribution medium and prior art-recognized equivalents and successor media, in which the software implementations of the present disclosure are stored.

Automatic Call or Contact Distribution (ACD) is a communication server software feature that processes incoming, outgoing, and internal calls and distributes them to groups of extensions called hunt groups or splits. The communication server also sends information about the operation of the ACD to the Call Management System (CMS) which stores and formats the data and produces real-time and historical reports on ACD activity. ACD is used by a contact center to route incoming calls to specifically assigned splits/skills and agents. ACD allows a system administrator to create an efficient call management environment.

The terms “determine”, “calculate,” and “compute,” and variations thereof, as used herein, are used interchangeably and include any type of methodology, process, mathematical operation or technique.

The term “module” as used herein refers to any known or later developed hardware, software, firmware, artificial intelligence, fuzzy logic, or combination of hardware and software that is capable of performing the functionality associated with that element. Also, while the present disclosure is described in terms of exemplary embodiments, it should be appreciated those individual aspects of the present disclosure may be separately claimed.

The terms “switch,” “server,” “contact center server,” or “contact center computer server” as used herein should be understood to include a Private Branch Exchange (PBX), an ACD system, an enterprise switch, or other type of telecommunications system switch or server, as well as other types of processor-based communication control devices such as, but not limited to, media servers, computers, adjuncts, and the like.

As used herein, the term “gateway” may generally comprise any device that sends and receives data between devices. For example, a gateway may comprise routers, switches, bridges, firewalls, other network elements, and the like, any and combination thereof.

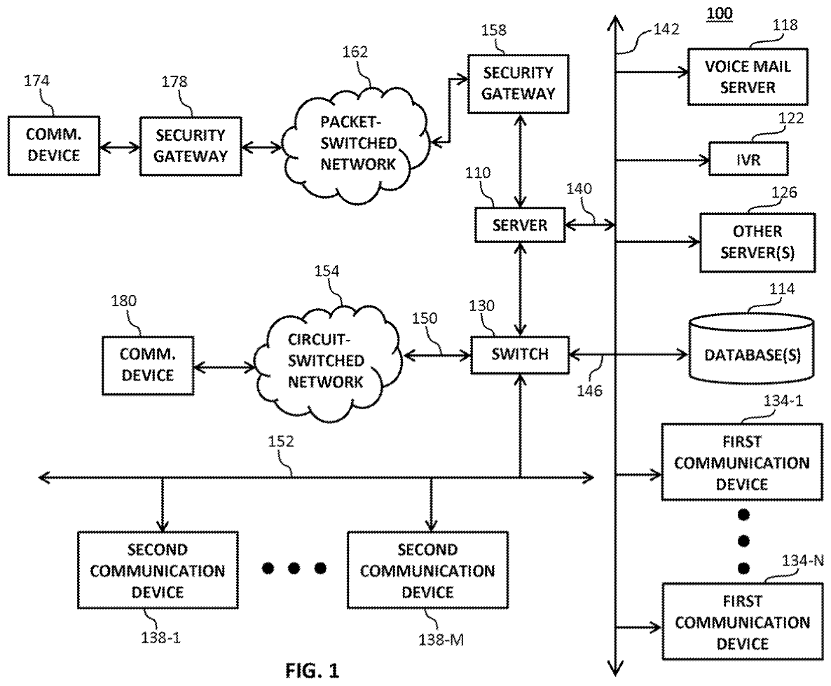

FIG. 1 shows an illustrative embodiment of a contact center 100 according to the present disclosure. The contact center 100 comprises a central server 110, a set of data stores or databases 114 within the contact center 100 containing contact or customer related information and other information that may enhance the value and efficiency of the contact processing, and a plurality of additional servers, namely a voice mail server 118, an Interactive Response unit (e.g., IVR) 122, and other servers 126, a switch 130, a plurality of working agents operating packet-switched (first) communication devices 134-1 to N (such as computer work stations or personal computers), and/or circuit-switched (second) communication devices 138-1 to M, all interconnected by a local area network LAN (or wide area network WAN) 142. The servers may be connected via an optional communication link 146 to the switch 130. The server 110 may, for example, be connected to the LAN 142 via the switch or via its own communication link 140. As will be appreciated, the other servers 126 may also include a scanner (which is normally not connected to the switch 130 or Web server), VoIP software, video call software, voice messaging software, an IP voice server, a fax server, a web server, an e-mail server, and the like. The switch 130 is connected via a plurality of trunks 150 to the Public Switch Telephone Network (PSTN) 154 and via link(s) 152 to second communication devices 138-1 to M. A gateway 158 is positioned between the server 110 and the packet-switched network 162 to process communications passing between the server 110 and the network 162. A gateway 158 may be Avaya Inc.'s, G700 Media Gateway™ and may be implemented as hardware such as via an adjunct processor (as shown) or as a chip in the server.

Although some embodiments are discussed with reference to a client-server architecture, it is to be understood that the principles of the present disclosure apply to other network architectures. For example, the present disclosure applies to peer-to-peer networks, such as those envisioned by the Session Initiation Protocol (SIP). In the client-server model or paradigm, network services and the programs used by end users to access the services are described. The client side provides a user with an interface for requesting services from the network, and the server side is responsible for accepting user requests for services and providing the services transparent to the user. By contrast, in the peer-to-peer model or paradigm, each networked host runs both the client and server parts of an application program. Additionally, the disclosure does not require the presence of packet- or circuit-switched networks.

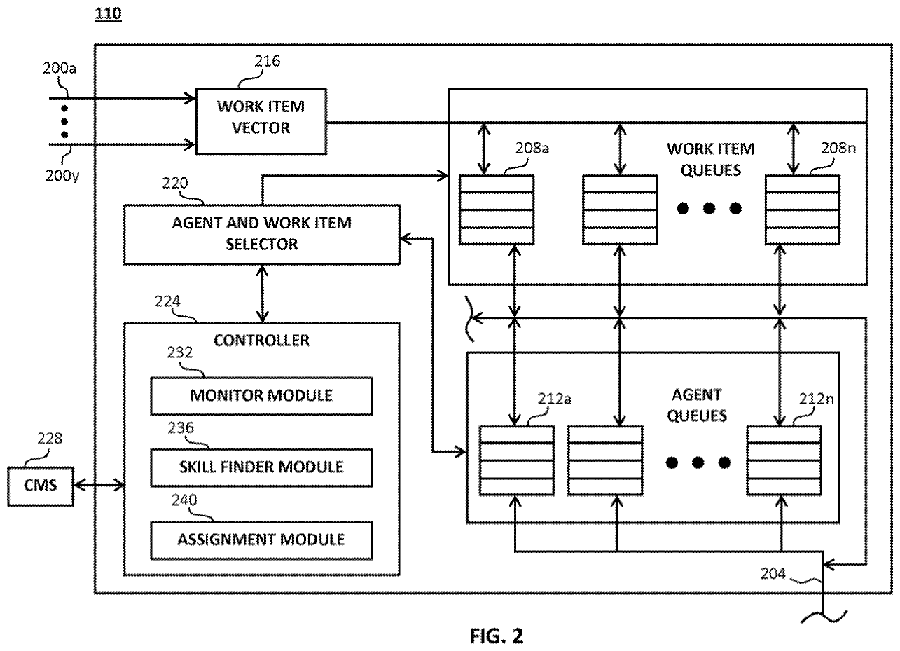

Referring to FIG. 2, one possible configuration of the central server 110 of a contact center is depicted. The server 110 is in communication with a plurality of customer communication lines 200a-y (which may be one or more trunks, phone lines, etc.) and agent communication line 204 (which may be a voice-and-data transmission line such as LAN 142 and/or a circuit switched voice line). The server 110 may include Avaya Inc.'s Operational Analyst™ (OA) with On-Line Analytical Processing (OLAP) technology or a Call Management System (CMS) 228 that gathers contact records and contact-center statistics for use in generating contact-center reports. OA and CMS will hereinafter be referred to jointly as CMS 228.

The switch 130 and/or server 110 may be any architecture for directing contacts to one or more agent communication devices. In some embodiments, the switch 130 may perform load-balancing functions by allocating incoming or outgoing contacts among a plurality of logically and/or geographically distinct contact centers. Illustratively, the switch and/or server may be a modified form of the subscriber-premises equipment sold by Avaya Inc. under the names Definity™ PBX-based ACD system, MultiVantage™ PBX, Communication Manager™, S8300™ media server and any other media servers, SIP Enabled Services™, Intelligent Presence Server™, and/or Avaya Interaction Center™, and any other products or solutions offered by Avaya or another company.

Typically, the switch/server is a stored-program-controlled system that conventionally includes interfaces to external communication links, a communications switching fabric, service circuits (e.g., tone generators, announcement circuits, etc.), memory for storing control programs and data, and a processor (i.e., a computer) for executing the stored control programs to control the interfaces and the fabric and to provide automatic contact-distribution functionality. Other types of known switches and servers are well known in the art and therefore not described in detail herein.

As may be seen in FIG. 2, included among the data stored in the server 110 is a set of work item queues 208a-n and a separate set of agent queues 212a-n. Each work item queue 208a-n corresponds to a different set of agent skills, as does each agent queue 212a-n. Conventionally, work items are prioritized and are either enqueued in individual ones of the work item queues 208a-n in their order of priority or in different ones of a plurality of work item queues 208a-n that correspond to a different priority. Likewise, each agent's skills are prioritized according to his or her level of expertise in that skill, and agents are either enqueued in individual ones of agent queues 212a-n in their order of expertise level or in different ones of a plurality of agent queues 212a-n that correspond to a skill and each one of which corresponds to a different expertise level.

Included among the control programs in the server 110 is a work item vector 216. Contacts incoming to the contact center are assigned by the work item vector 216 to different work item queues 208a-n based on a number of predetermined criteria, including customer identity, customer needs, contact center needs, current contact center queue lengths, customer value, and the agent skill that is required for the proper handling of the contact. Agents who are available for handling work items are assigned to agent queues 212a-n based on the skills that they possess. An agent may have multiple skills, and hence may be assigned to multiple agent queues 212a-n simultaneously. Furthermore, an agent may have different levels of skill expertise (e.g., skill levels 1-N in one configuration or merely primary skill levels and secondary skill levels in another configuration), and hence may be assigned to different agent queues 212a-n at different expertise levels.

In one configuration, the contact center may be operated by a contract operator, and each of the work item queues 208a-n, and possibly each of the agent queues 212a-n, corresponds to a different client. Each client may have a separate service level agreement or other type of performance measurement agreement with the contract operator regarding performance expectations, goals, requirements or specifications for the client's respective queue(s). The service level agreement may set forth penalties, such as financial penalties, for failing to comply with the service level agreement's requirements.

With reference to FIG. 1, the first agent communication devices 134-1, . . . 134-N are packet-switched and may include, for example, IP hardphones such as the 4600 Series IP Phones™ by Avaya Inc., IP softphones such as the IP Softphone™ by Avaya Inc., Personal Digital Assistants (PDAs), Personal Computers (PCs), laptops, packet-based H.320 video phones and conferencing units, packet-based voice messaging and response units, packet-based traditional computer telephony adjuncts, peer-to-peer based communication devices, and any other suitable communication device. The second communication devices 138-1, . . . 138-M are circuit-switched and each correspond to one of a set of internal extensions Ext1, . . . ExtM, respectively. The second communication devices may include, for example, wired and wireless telephones, PDAs, H.320 videophones and conferencing units, voice messaging and response units, traditional computer telephony adjuncts, and any other suitable communication device.

The packet-switched network 162 may be any data and/or distributed processing network, such as the Internet. The network 162 typically includes proxies (not shown), registrars (not shown), and routers (not shown) for managing packet flows. The packet-switched network 162 is in communication with a first external communication device 174 via a gateway 178, and the circuit-switched network 154 with a second external communication device 180. In one configuration, the server 110, network 162, and first communication devices 134 are SIP compatible and may include interfaces for various other protocols such as the Lightweight Directory Access Protocol or LDAP, H.248, H.323, Simple Mail Transfer Protocol or SMTP, IMAP4, ISDN, E1/T1, and analog line or trunk.

It should be noted that the present disclosure does not require any particular type of information transport medium between a switch or server and first and second communication devices, i.e., the present disclosure may be implemented with any desired type of transport medium as well as combinations of different types of transport channels. It should also be emphasized that the configuration of the switch, server, communication devices, and other elements as shown in FIG. 1 is for purposes of illustration only and should not be construed as limiting the disclosure to any particular arrangement of elements.

The central server 110 may be notified via LAN 142 of an incoming work item by the communications component (e.g., switch 130, fax server, e-mail server, web server, and/or other server) receiving the incoming work item. The incoming work item is held by the receiving telecommunications component until the server 110 forwards instructions to the component to forward or route the contact to a specific contact center resource, such as the IVR unit 122, the voice mail server 118, and/or first or second communication device 134, 138 associated with a selected agent.

The server 110 distributes and connects these work items to communication devices of available agents based on the predetermined criteria noted above. When the central server 110 forwards a voice contact (or first work item) to an agent, the central server 110 also forwards customer-related information from databases 114 to the agent's computer work station for viewing (such as by a pop-up display) to permit the agent to better serve the customer. Depending on the contact center configuration, the central server 110 may forward a list of work items to an available agent to preview before forwarding the work item itself and the data associated therewith to the agent. The agents process the work items or contacts sent to them by the central server 110. This embodiment is particularly suited for a Customer Relationship Management (CRM) environment in which customers are permitted to use any media to contact a business. In a CRM environment, both real-time and non-real-time contacts must be handled and distributed with equal efficiency and effectiveness.

According to an embodiment of the present disclosure, included among the programs executing on the server 110 are an agent and work item selector 220 and a controller 224, as shown in FIG. 2. The selector 220 and the controller 224 are stored either in the main memory or in a peripheral memory (e.g., disk, CD ROM, etc.) or some other computer-readable medium of the contact center 100. The selector 220 and the controller 224 collectively effect an assignment between available work items (or contacts) and available agents in a way that tends to maximize contact center efficiency for the current contact center state. The selector 220 uses predefined criteria in selecting an appropriate agent to service the work item. The controller 224 assists the work item vector 216 in routing the contacts to the appropriate work item queue 208 and the selector 220 in routing a contact to a most desirable agent in the agent queue 212. The controller 224, in particular, obtains, for each of a plurality of contact center goals or objectives, status information, or information respecting whether or not a corresponding goal (for example, the service level agreement) is being met or unmet and/or a level to which the corresponding goal is being met or unmet.

Agent and customer profiles are typically maintained and updated by a profile generator (not shown). Upon the completion of handling a work item, the generator may collect selected metrics for the work item. These metrics include the skill involved in servicing the work item, the identifier of the servicing agent, the contact duration, the transaction or contact type (e.g., catalog sale, information request, complaint, etc.), the time-of-day, the result (e.g., the type of sale, the number of units sold, revenue generated, service ticket closure or escalation, the information provided, etc.), a self-rating of the servicing agent respecting the agent's proficiency in handling the work item, the rating of the customer of the agent's proficiency in handling the work item, the rating of another party, such as the agent's supervisor or another observer, of how the work item was serviced, whether the agent requested assistance, and whether the agent's training was completed, and stores the information in the database 114, such as CMS 228. The metrics over a selected period of time are typically stored under each agent's profile and may include metrics associated with a plurality of work items serviced by the agent for each agent skill. Each agent's profile may also contain his or her skills and/or level(s) of skill expertise. Each customer profile includes metrics associated with a plurality of contacts by the customer.

The controller 224 has access to various performance measures of each of the agent resources and the contact handling history of the resources in the system. The controller 224 is also configured to use various systems, known in the art, to assess the performance of individual agent resources in processing work items. The controller 224 is further configured to find an area of competency for each of the agent resources based on their performance measures and contact handling history. The controller 224 is further configured to utilize such findings in maximizing contact center efficiency.

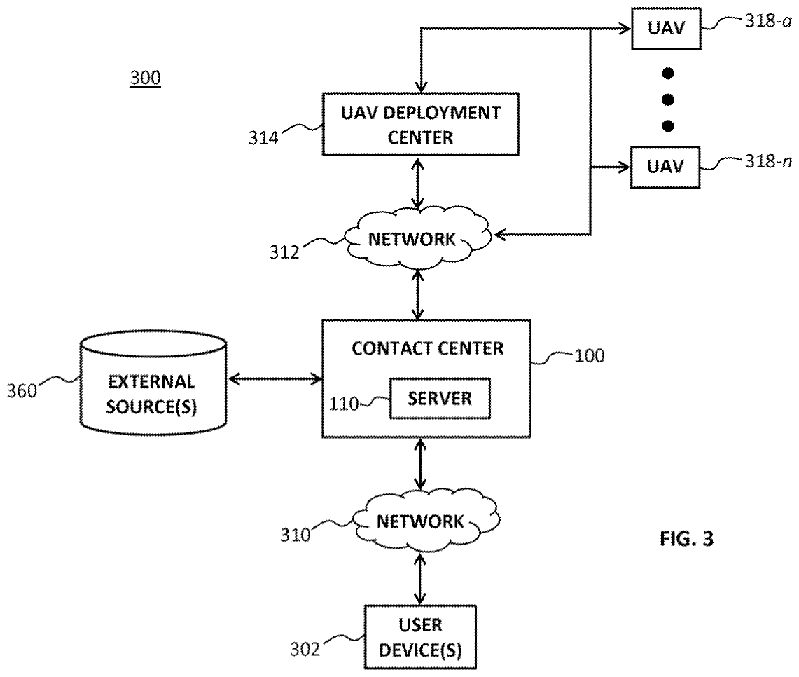

FIG. 3 illustrates an exemplary system and communication architecture 300 in accordance with the present disclosure. A contact center 100 comprising a central server 110 receives communications from one or more user communication devices 302 via a network 310. The user communication device(s) 302 may correspond to the first and second communication devices 174, 180 shown in FIG. 1 and may represent, for example, a customer's or user's cell phone or smart phone, e-mail system, tablet, desktop or laptop computer, or other device that allows the user to interact with the contact center 100. The communication device 302 may also include, for example, a transmitter that is connected to one or more sensors or systems associated with an alarm or monitoring system (e.g., motion, heat, smoke, chemical, and/or moisture detectors, cameras, etc.) and transmits a signal to the contact center 100 indicating the potential occurrence of an actionable event such as an unauthorized entry, a fire, flooding, etc. The network 310 may include, for example, a LAN, a WAN, the Internet, a cellular telephone network, or any other suitable type of communication system.

The contact center 100 may also be in communication with, and receive information from, one or more external sources 360 such as databases, subscription services providing information and updates regarding the weather, air and ground traffic, crime alerts, etc., and the like. These sources 360 are external to the contact center 100 and may transmit information to the contact center 100 via a network (not shown) similar to network 310. In addition, the server 110 may retrieve information from one or more databases 114 within the contact center 100, as shown in FIG. 1, which may include, for example, user or customer account information. The contact center 100 communicates with a UAV deployment center 314 via a network 312, which may be similar to the network 310 and may share some of the same communication infrastructure as the network 310. The deployment center 314 manages and maintains a fleet of UAVs 318a-n and provides information to the contact center 100 regarding the features and status of each UAV 318a-n.

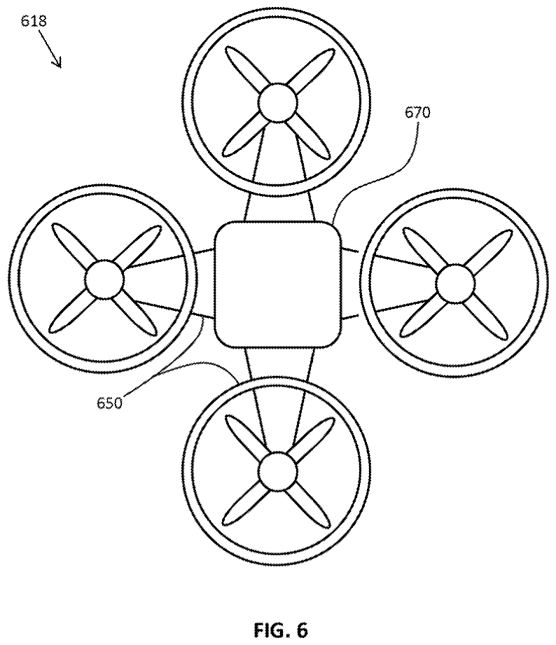

As is known in the art, the UAVs 318a-n may include a variety of configurations that allow them to perform in various environments. For example, the UAV may comprise a fixed wing design or a rotary airfoil type UAV with one or more rotors, such as quadcopter, hexarotor, and multicopter designs. FIG. 6 is a simplified illustration of a UAV 618 with a quadcopter design comprising a body 650 and a central compartment 670. The UAV 618 may include a variety of sensors and sensing systems (not labeled) mounted in or on the body 650 and/or the central compartment 670 to, for example, determine the UAV's speed, altitude and attitude, measure environmental conditions such as wind speed and barometric pressure, and detect the presence of obstacles in the flightpath, including other UAVs. The UAV 618 may also include one or more communications systems (not labeled) that may be mounted in or on the body 650 and/or the central compartment 670. For example, the UAV 618 may include a wireless communication system that permits the UAV 618 to transmit information gathered by the one or more sensors or sensing systems back to the contact center 100 and/or the UAV deployment center 314. The UAV 618 may further comprise a variety of audio and/or visual communication equipment and systems (not labeled) mounted in or on the body 650 and/or central compartment 670 such as one or more cameras, video display screens, speakers, and microphones that permit communication between the agent and the user or other people nearby.

In addition, each of the UAVs 318a-n includes one or more processors (not shown) in communication with one or more computer-readable storage media (not shown) containing executable instructions related to operation and maintenance of the UAV. With reference to FIG. 6, the processor(s) and storage media may be mounted within the central compartment 670 of the UAV 618. The UAVs 318a-n may be fully or partially autonomous and may use a variety of pre-programmed guidance, navigation, and control systems that utilize global positioning systems (GPS), navigation waypoints, and the like to determine the UAV's current location and autonomously navigate to a target destination. The UAVs 31 8a-n may also be fully or partially controllable by a remote pilot who controls the UAV using commands sent via a wireless link (not shown).

One or more of the UAVs 318a-n may be specially equipped to handle particular situations. For example, a UAV deployed to handle a medical emergency, e.g. UAV 618 shown in FIG. 6, may have one or more compartments, e.g. central compartment 670, containing a cargo or payload, which may comprise, for example, medical supplies such as medication, equipment such as a defibrillator, a first aid kit, etc. that may be provided to the user or other personnel at a destination at which the UAV arrives. The UAV may also include onboard equipment such as a camera and video display screen to allow an agent at the contact center 100 to visually assess a user's medical condition and provide medical assistance and instructions and/or a user interface that allows the user to communicate with the UAV and/or the agent.

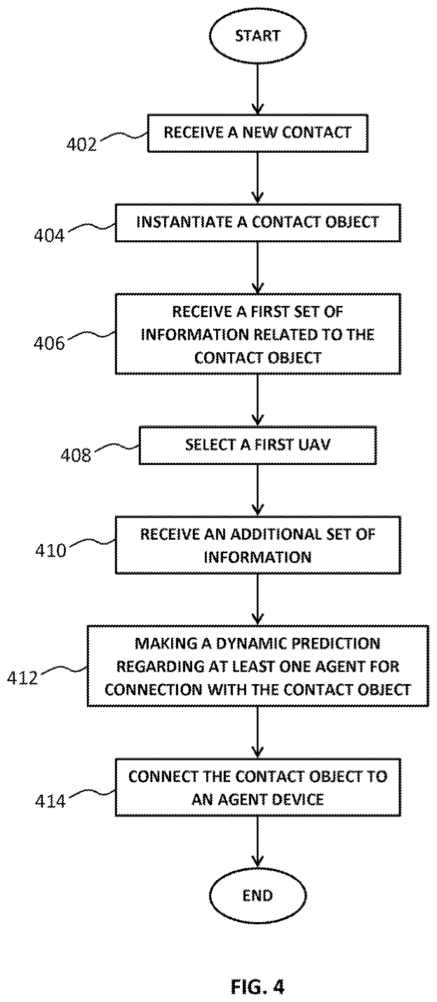

The flowchart of FIG. 4 illustrates an exemplary method for allocating agent resources within a contact center in accordance with the present disclosure. This method, and all methods disclosed herein, may be performed all or in part by a server of a contact center 100, e.g. central server 110, executing instructions stored in one or more memory devices.

With reference to FIG. 4, the method begins at Step 402 when the contact center receives a new contact, in which the new contact comprises a communication from a user. The communication may relate to a request for assistance from a user, and as described herein, the user may be the person requesting assistance or a person requesting assistance on behalf of a third party. In some embodiments, the user may be a non-human entity such as an automated monitoring or alarm system that utilizes various sensors to detect, for example, a potential unauthorized entry, and the contact may comprise a signal sent to the contact center by the automated system.

At Step 404, a contact object corresponding to the new contact is instantiated. At Step 406, a first set of information related to the contact object is received by the contact center. The first set of information may comprise, for example, information received from the user, including one or more of a type of communication or request (e.g., a request for medical assistance, notification that a crime has potentially been committed, a motion sensor alarm, etc.), a priority level of the request (e.g., a life-threatening event such as a heart attack or stroke, as opposed to a minor injury), and a target location or destination (e.g., a street address, GPS coordinates, etc.). The target destination may comprise the user's current location, which may be provided automatically to the contact center by the user's communication device, or the target destination may comprise a second location specified by the user. The target destination may also comprise, for example, a location of the motion sensor that generated an alarm, which may be included in the signal provided to the contact center.

The first set of information may further comprise data retrieved from one or more databases within the contact center and/or information received from one or more external sources. The information retrieved from the database(s) within the contact center may comprise user account information such as a user's contact information and medical history, and if the user is a member of a subscription-based service, the account information may include, for example, the user's service level and benefits. The information retrieved from the external source(s) may comprise, for example, current environmental conditions at the target destination and along one or more routes or flightpaths to the target destination, e.g., wind speed and direction, current rainfall, weather forecasts, and any other conditions that might affect the arrival time of the UAV.

In one embodiment, receiving the first set of information may comprise connecting the user to a human agent within the contact center, after which the user may provide the first set of information to the agent. The agent may be selected in accordance with the disclosed methods for assignment of contacts within a contact center. In another embodiment, the user may provide some or all of the first set of information via an automatic system, such as a voice recognition system, e.g. IVR unit 122 in FIG. 1. For example, in response to questions or prompts by the IVR system, the user may indicate verbally, or by pressing one or more buttons on his or her communication device, whether the request is “medical” or “non-medical,” whether the request relates to a life-threatening situation, what type of assistance is desired, etc.

Based on the first set of information, at least one UAV is selected at Step 408 for deployment to the target destination. Dispatch instructions are sent to the selected UAV(s), including the target destination associated with the new contact. Selection of the UAV may involve communication between the contact center and a UAV deployment center that manages and maintains a fleet of UAVs. The UAV(s) may also be selected based on one or more features of each UAV. The UAV features may comprise, for example, a current GPS location, a current battery condition and/or fuel level, maintenance status, top speed, payload capacity and type, UAV configuration and onboard equipment, etc. For example, a user requesting assistance for a life-threatening medical emergency in a remote location will require a UAV that is able to quickly travel the required distance and is equipped to handle the emergency. In addition, the situation may require deployment of one specialized UAV, e.g., a UAV carrying medical equipment, and one or more additional general purpose UAVs, which may, for example, provide additional illumination of the scene, remote monitoring, etc. The UAV and/or payload may be selected based on, for example, one or more of a specific request by the user, a request by an agent of the contact center, instructions contained in a service agreement, or historical data regarding similar types of contacts handled by the contact center in the past. One or more attributes or characteristics of the selected UAV(s) may be associated with the contact object. For example, the characteristic may comprise a projected arrival time of the UAV(s) at the target destination.

At Step 410, one or more additional sets of information may be received by the contact center at Step 410. In some embodiments, the contact center may remain in communication with, and/or periodically receive information from, the user, the deployment center, and/or the deployed UAV(s) during a travel period of the deployed UAV(s) to the target destination. The additional set(s) of information may relate to one or more time varying attributes of the user and/or the UAV(s), i.e. an attribute or characteristic that changes in the time between when the one or more UAVs are dispatched to the target destination and when the UAV(s) arrive at the target destination. The time-varying attribute(s) may comprise, for example, one or more characteristics of the UAV(s), such as an updated GPS location and/or battery condition of the deployed UAV(s), one or more attributes of the user, such as an updated GPS location of the user or updated GPS coordinates of the target destination, or a change to the request (e.g., a change in the user's health condition), and other information received from the UAV(s), such as changes in wind speed and other environmental conditions. The UAV(s) may transmit the additional set(s) of information to the contact center.

In other embodiments, the user may disconnect from the contact center if, for example, the request is lower priority (i.e. non-critical) and/or if the expected flight time of the UAV is lengthy. Upon arrival of the UAV(s) at the target destination, the user may reconnect to the contact center via a user communication device (a “direct” connection) or the user may be connected to the contact center via the audio and/or visual capabilities of the UAV(s) (an “indirect” connection).

The contact center may also remain in communication with, and/or periodically receive information from, one or more of the database(s) within the contact center and the external source(s). The additional set(s) of information may comprise, for example, an updated environmental condition at the target destination or along a route to the target destination. In addition, the contact center may receive additional information related to one or more additional UAVs. For example, the contact center may receive information regarding a GPS location, battery condition, and features of one or more additional UAVs at the deployment center and/or near the target destination or along the flightpath to the target destination. This information may be used, for example, to select one or more additional UAVs for deployment to the target destination and/or to ensure that the flightpath to the target destination is clear of other UAVs. The contact center may transmit the additional set(s) of information to one or more of the user, the UAV(s), and the deployment center.

At step 412, the contact center server makes one or more dynamic predictions regarding at least one particular agent for connection with the contact object. At one or more points during the excursion of the UAV(s), at least one human agent of the contact center may need to be connected to the contact object, for example, to communicate with the user, provide instructions to the UAV(s), etc. Based on the first set of information and the additional set(s) of information, the dynamic prediction made at Step 412 may comprise a prediction of one or more particular agents within the contact center that should be connected to the contact object. Because the particular agent(s) will typically need to possess a specific skill or set of skills, the dynamic prediction may also be made based on one or more attributes of the particular agent(s). These attributes may include, for example, an agent's education, training, level of experience, languages spoken by the agent, and the like. As described herein, one or more of these attributes may be stored in an agent profile, and the agent profile may be matched with the needs of the user as disclosed herein. For example, the particular agent(s) may comprise one or more of a medical professional (a medical doctor, physician assistant, nurse, etc.), a person trained to remotely pilot the UAV, or a person knowledgeable about a particular product. The agent's attributes may also include his or her current status, such as a current number of queued contacts, the agent's stress level (e.g., has the agent recently worked through a scheduled break), etc.

In some embodiments, the dynamic prediction at Step 412 may also involve dynamically predicting an optimal time period in which to connect the contact object to the agent communication device(s) associated with the particular agent(s). The optimal time period may comprise, for example, a time period just prior to, or upon, arrival of the UAV(s) at the target destination and may be calculated using, for example, the distance between the target destination and the UAV's current location, the UAV's top speed, the current environmental conditions, etc. The optimal time period may be adjusted during the travel period of the UAV(s) based on one or more of the first set of information and the additional set(s) of information. For example, the optimal time period may be adjusted based on one or more time-varying attributes of the user and/or the UAV(s), such as movement of the user to a new target destination, as well as other information such as changing environmental conditions along the flightpath to the target destination, the presence of an obstacle in the flightpath, etc.

Following the dynamic prediction(s) at Step 412, the contact object is connected at Step 414 to an agent device associated with each particular agent, after which the method concludes. For example, the contact center may transmit communication instructions to the UAV(s) so that an active communication link is created between the user and the agent so that the agent is able to communicate with the user or with people nearby via audio and/or visual communication equipment on the UAV(s). In some embodiments, the agent may also be connected to the UAV(s) so that the agent is able to remotely pilot the UAV, deploy a payload that is onboard the UAV, etc.

Skilled agents represent a scarce resource within the contact center 100, and in most cases, the skilled agent will be required only at certain time periods during the UAV's excursion. Thus, each agent's time and availability should be optimized to ensure that agents with the appropriate skills are available for connection with each contact object at the optimal time. In performing the dynamic prediction at Step 412, the server of the contact center may treat each deployed UAV as an additional connection of the contact object and may prioritize and manage each incoming work item, or contact object, as described with respect to FIG. 2, based at least in part on a deployed UAV that is considered to be a connection of the contact object. For example, a contact object may have multiple connections that change over time. Initially, the contact object may be connected with an IVR system. The contact may then be connected with an agent of the contact center and may also be connected with yet another agent of the contact center at one or more points during a communications session. The deployed UAV may also be considered as one connection of the contact object even though the user and the UAV may not actually communicate with one another.

As described more fully herein, by predicting when the UAV will reach the target destination, the contact center may dynamically determine when to connect the contact object with a skilled medical agent, or some other skilled agent(s). When the skilled agent(s) and the contact object are connected, the connection capabilities between the user, the UAV, and/or the contact center may change, such as establishing one or more physical communication channels between a device of the agent(s) and a user device and/or communication devices on the one or more UAVs that were deployed and have arrived at the target destination. For example, each incoming contact may be associated with a respective contact object, which may then be handled by other systems of the contact center 100. As described with respect to FIG. 2, the work item vector 216, the agent and work item selector 220, and the controller 224 may work together to:
A) select one or more appropriate UAVs to deploy;
B) add the selected UAV(s) as a connection of the contact object; and
C) assign the contact object to a work item queue 208a-n and select one or more agents with the appropriate skills.
Thus, the contact object will have an attribute that represents when the deployed UAV is expected to arrive at the target destination.

As described in item (C) above, the selector 220 and the controller 224 also work together to route the contact object to the appropriate agent queue 212a-n, such that the selected agent(s) may be connected with the contact object at the optimal time period as described herein. This situation is similar to an initial contact in a contact center that has been queued back into the contact center for re-routing to a different agent with a specialized skillset. However, the attributes related to UAV arrival time associated with these queued contact objects are changing dynamically, and therefore the paradigm is slightly different from a regular queued contact object. In the regular case, the selection criteria comprise “the most suitable agent as soon as possible,” whereas in the disclosed UAV scenario, the selection criteria comprise “the most suitable agent at a specific future time, the values of which are being continuously evaluated based on an ongoing input stream of changing attributes.”

For example, in a large contact center with hundreds of incoming calls, the agent device associated with an agent may include a display that provides the agent with a view of the current contact that the agent is handling, along with a list of queued contact objects, which may include the predicted arrival time of the UAV(s) associated with each contact object and the priority of the contact. Thus, the agent could be handling one contact with a first user at a first target destination, while waiting for a second UAV associated with a second queued contact object to arrive at a second target destination. If the arrival of the second UAV at the second target destination is delayed, the agent may move to the next item in his or her queue. By considering the projected arrival time of the associated UAV(s) as an attribute of queued contact objects, multiple time varying attributes may be used to optimize and predict the assignment and connectivity of skilled agents to the contact objects with associated UAVs as those associated UAVs approach, or arrive at, their target destinations.

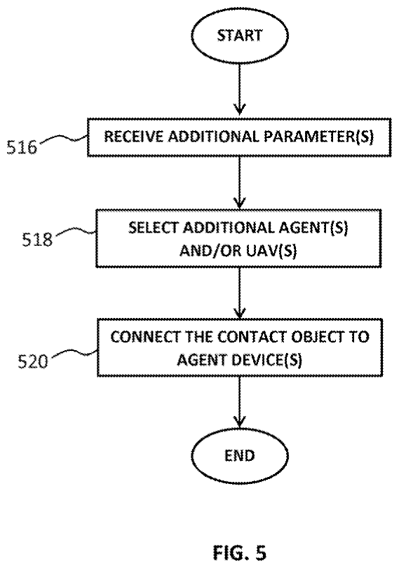

The flowchart of FIG. 5 illustrates an exemplary method for receiving additional information following arrival of the one or more UAVs at the target destination. The method begins with the arrival of the UAV(s) at the target destination, and at Step 516, one or more additional parameters may be received by the contact center upon or following arrival of the UAV(s) at the target destination. The parameter(s) may relate to one or more of the user or contact object, a target destination condition, and a condition of the UAV(s) at the target destination. For example, a sudden deterioration in the user's health condition may occur, which may require connection of an additional agent or an agent with a different skillset. In addition, the environmental conditions at the target destination may change (e.g., ambient light is reduced due to the sun setting) or one of the deployed UAVs may have a low battery, which may require deployment of one or more additional UAVs to the target destination. Based on these one or more additional parameters, at Step 518, one or more additional agents may be selected for connection with the contact object and/or one or more additional UAVs may be selected for deployment to the target destination. The additional agent(s) and/or UAV(s) may be selected as previously described, in which the selection may be based, respectively, on attributes of the agent(s) and features of the UAV(s). The selection may also be based on the first set of information and/or the additional set(s) of information. At Step 520, the contact object is connected to an agent device corresponding to each of the one or more additional agents, after which the method concludes.

The presently disclosed system and method may be more fully understood by way of the following examples.

### EXAMPLE 1

### A Medical Emergency

A user who witnessed a nearby person suffering an apparent heart attack dials an emergency number and is connected to a contact center 100. The central server 110 of the contact center recognizes this communication from the user as a contact and instantiates a contact object that will be associated the contact. The user may follow a series of prompts from the contact center, e.g., from IVR system 122, or the user may be connected to a human agent of the contact center. The user indicates that the issue type is “potential cardiac arrest,” the priority of the call is “high,” and the service requested is delivery of a defibrillator to the address of 123 Maple Street. Based on this information, the server queries the inventory of available UAVs at the deployment center 314 and selects a UAV with the appropriate features to meet the user's request. For example, the selected UAV is equipped with, or is capable of delivering, a defibrillator and is one of the fastest models available. The selected UAV is then dispatched from the deployment center to the target destination.

Based on this initial set of information, the contact center server dynamically predicts that the journey to the target destination will take approximately 10 minutes and that an agent skilled in cardiovascular emergencies, e.g. an emergency medical technician (EMT), should be connected to the contact object shortly before the selected UAV arrives at the target destination, e.g., at between eight and nine minutes after the selected UAV departed the deployment center. With reference to the exemplary contact center 100 in FIG. 1, a work item vector 216, an agent and work item selector 220, and a controller 224 within the server work together to assign the contact object, or work item, to a work item queue 208, select an agent who is a licensed EMT from one of the agent queues 212, and route the contact object to that agent.

During the flight of the selected UAV to the target destination, the contact center remains in communication with the user and continues to receive information from several sources. Prior to the arrival of the selected UAV, the user indicates that the person seeking medical assistance has been moved to a new location that is a shorter distance away from the present location of the selected UAV. In addition, the contact center receives information that the weather conditions along the route to the target destination reflect a decrease in wind speed. The contact center relays this information to the selected UAV, and the server uses the information to dynamically update the arrival time of the UAV to, for example, 4-6 minutes, and correspondingly, the time period during which the EMT should be connected to the contact object. The server also uses each new piece of information to update the work item and/or agent queue assignment and the specific agent selection as needed to ensure that an agent with the appropriate skills is available for connection to the contact object at the appropriate time. For example, if the originally selected EMT is unavailable for connection at the optimal time period associated with the updated arrival time, the contact center may select a different EMT for connection with the contact object as described herein.

The EMT is connected with the contact object at the appropriate time and communicates with the user via a video display screen and microphone on the UAV to obtain information regarding the medical condition of the person needing medical attention, to provide instructions regarding use of the defibrillator, etc. Following arrival of the UAV at the target destination, the user indicates to the contact center that additional lighting is needed. The contact center may use this additional information to select and dispatch a second UAV from the deployment center to the target destination to provide additional lighting, as previously described.

### EXAMPLE 2

### A Potential Unauthorized Entry

In another embodiment, one or more UAVs may be used to monitor a large premises such as a warehouse and the surrounding property. The UAVs may be associated with a contact center that monitors an alarm system at the premises and may autonomously roam around the premises or may be deployed only upon detection of an actionable event. For example, a motion sensor associated with the alarm system detects movement in an unauthorized area and sends a signal to the contact center, which deploys two UAVs to the target destination, i.e. the area near the motion sensor. In some instances, the signal may be sent to personnel at the premises, who then initiate contact with the contact center.

Using the optimization techniques described herein, one or more skilled security agents may be connected to the UAVs at one or more times during the UAVs' excursion. For example, one agent may use the first UAV's onboard equipment such as a camera and lights to scan the area for property damage. The same agent or a different agent may use the second UAV for surveillance and additional lighting or to locate and follow a potential intruder. The UAV(s) may include microphones to broadcast an alarm or audible warning to persons in the area and/or flashing lights to deter entry into the area. In addition, when there are a large number of deployed UAVs and associated security personnel, the contact center monitors one or more time-varying attributes associated with each contact object as described herein to, for example, redirect one or more UAVs to a different location (e.g. if a higher priority event occurs), deploy one or more new UAVs to the original location, and update the dynamic predictions regarding the selected agent(s) for each UAV and the optimal time period for connecting the agent(s). As a result, the contact center is able to ensure that agents are available for connection within the optimal time period and that use of each agent's time and skills is optimized.

### EXAMPLE 3

### Request for Directions and Product Information

In a further embodiment, UAVs may be used in a large commercial setting such as a shopping mall, event center, or hotel to provide information to guests. As described in the previous example, the UAVs may roam around the premises or may be deployed in response to a specific request. For example, an event attendee is lost and requests assistance finding a particular location. Using the techniques described herein, an agent with knowledge of the layout of the event center is connected with the UAV upon arrival at the attendee's location and may provide audible instructions, a map of the event center via a video display screen, etc. In another example, a customer at a shopping mall has a question about a particular product and scans a product code with his or her smart phone. Using the techniques described herein, a UAV is deployed to the customer's location and the customer is connected to an agent who is knowledgeable about that product. In other examples, the UAVs and their associated agents may be used in these types of locations as “tour guides” to provide information about upcoming events, directions to local attractions, and the like. As described herein, the agents may be associated with a contact center that monitors one or more time-varying attributes associated with each contact object to update the dynamic predictions regarding the selected agent(s) for each UAV and the optimal time period for connecting the agent(s), thereby ensuring that agents are available for connection within the optimal time period and maximizing and optimizing the use of each agent's time and skills.

Other non-limiting examples in which the present systems and methods may be used include deployment of one or more UAVs in response to a customer's request for roadside assistance, e.g., delivery of fuel or providing troubleshooting, or in response to a customer's request for personal security, e.g., accompanying a person to his or her car at night. In these examples, the user may subscribe to a service that provides UAVs and agents in accordance with a service agreement. In addition, UAVs may be deployed in response to a request for assistance by law enforcement personnel and/or in response to a report of a potential crime in progress or a crime that has been committed.

The flowchart and block diagrams in the Figures illustrate the architecture, functionality, and operation of possible implementations of systems, methods and computer program products according to various aspects of the present disclosure. In this regard, each block in the flowchart or block diagrams may represent a module, segment, or portion of code, which comprises one or more executable instructions for implementing the specified logical function(s). In addition, while the flowcharts have been discussed and illustrated in relation to a particular sequence of events, it should be appreciated that changes, additions, and omissions to this sequence may occur without materially affecting the operation of the disclosure. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, may be implemented by special purpose hardware-based systems that perform the specified functions or acts, or combinations of special purpose hardware and computer instructions.

As will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “circuit,” “module,” “component,” or “system.” Furthermore, aspects of the present disclosure may take the form of a computer program product embodied in one or more computer readable media having computer readable program code embodied thereon.

Any combination of one or more computer readable media may be utilized. The computer readable media may be a computer readable signal medium or a computer readable storage medium. A computer readable storage medium may be, for example, but not limited to, an electronic, magnetic, optical, electromagnetic, or semiconductor system, apparatus, or device, or any suitable combination of the foregoing. More specific examples (a non-exhaustive list) of the computer readable storage medium would include the following: a portable computer diskette, a hard disk, a random access memory (RAM), a read-only memory (ROM), an erasable programmable read-only memory (EPROM or Flash memory), an appropriate optical fiber with a repeater, a portable compact disc read-only memory (CD-ROM), an optical storage device, a magnetic storage device, or any suitable combination of the foregoing. In the context of this document, a computer readable storage medium may be any tangible medium that may contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including but not limited to wireless, wireline, optical fiber cable, RF, etc., or any suitable combination of the foregoing.

Computer program code for carrying out operations for aspects of the present disclosure may be written in any combination of one or more programming languages, including an object oriented programming language such as JAVA, SCALA, SMALLTALK, EIFFEL, JADE, EMERALD, C++, CII, VB.NET, PYTHON or the like, conventional procedural programming languages, such as the “c” programming language, VISUAL BASIC, FORTRAN 2003, PERL, COBOL 2002, PHP, ABAP, dynamic programming languages such as PYTHON, RUBY, and GROOVY, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a LAN or WAN, or the connection may be made to an external computer (for example, through the Internet using an Internet Service Provider) or in a cloud computing environment or offered as a service such as a Software as a Service (SaaS).

Aspects of the present disclosure are described herein with reference to flowchart illustrations and/or block diagrams of methods, apparatuses (systems) and computer program products according to embodiments of the disclosure. It will be understood that each block of the flowchart illustrations and/or block diagrams, and combinations of blocks in the flowchart illustrations and/or block diagrams, may be implemented by computer program instructions. These computer program instructions may be provided to a processor of a general purpose computer, special purpose computer, or other programmable data processing apparatus to produce a machine, such that the instructions, which execute via the processor of the computer or other programmable instruction execution apparatus, create a mechanism for implementing the functions/acts specified in the flowchart and/or block diagram block or blocks. Alternatively, a symmetric multiprocessor (SMP) system or other configuration including a plurality of processors may be used.

These computer program instructions may also be stored in a computer readable medium that when executed may direct a computer, other programmable data processing apparatus, or other devices to function in a particular manner, such that the instructions when stored in the computer readable medium produce an article of manufacture including instructions which when executed, cause a computer to implement the function/act specified in the flowchart and/or block diagram block or blocks. The computer program instructions may also be loaded onto a computer, other programmable instruction execution apparatus, or other devices to cause a series of operational steps to be performed on the computer, other programmable apparatuses or other devices to produce a computer implemented process such that the instructions that execute on the computer or other programmable apparatus provide processes for implementing the functions/acts specified in the flowchart and/or block diagram block or blocks.

While the exemplary embodiments illustrated herein show the various components of the system collocated, certain components of the system may be located remotely, at distant portions of a distributed network, such as a LAN and/or the Internet, or within a dedicated system. Thus, it should be appreciated, that the components of the system may be combined in to one or more devices, such as a switch, server, and/or adjunct, or collocated on a particular node of a distributed network, such as an analog and/or digital telecommunications network, a packet-switch network, or a circuit-switched network. It will be appreciated from the preceding description, and for reasons of computational efficiency, that the components of the system may be arranged at any location within a distributed network of components without affecting the operation of the system. For example, the various components may be located in a switch such as a PBX and media server, gateway, in one or more communications devices, at one or more users' premises, or some combination thereof. Similarly, one or more functional portions of the system could be distributed between a telecommunications device(s) and an associated computing device.

Furthermore, it should be appreciated that the various links connecting the elements may be wired or wireless links, or any combination thereof, or any other known or later developed element(s) that is capable of supplying and/or communicating data to and from the connected elements. These wired or wireless links may also be secure links and may be capable of communicating encrypted information. Transmission media used as links, for example, may be any suitable carrier for electrical signals, including coaxial cables, copper wire, and fiber optics, and may take the form of acoustic or light waves, such as those generated during radio-wave and infra-red data communications.

A number of variations and modifications of the disclosure may be used. It would be possible to provide for some features of the disclosure without providing others. For example, in one alternative embodiment, the systems and methods of this disclosure may be implemented in conjunction with a special purpose computer, a programmed microprocessor or microcontroller and peripheral integrated circuit element(s), an ASIC or other integrated circuit, a digital signal processor, a hard-wired electronic or logic circuit such as discrete element circuit, a programmable logic device or gate array such as PLD, PLA, FPGA, PAL, special purpose computer, any comparable means, or the like. In general, any device(s) or means capable of implementing the methodology illustrated herein may be used to implement the various aspects of this disclosure. Exemplary hardware that may be used for the present disclosure includes computers, handheld devices, telephones (e.g., cellular, Internet enabled, digital, analog, hybrids, and others), and other hardware known in the art. Some of these devices include processors (e.g., a single or multiple microprocessors), memory, nonvolatile storage, input devices, and output devices. Furthermore, alternative software implementations including, but not limited to, distributed processing or component/object distributed processing, parallel processing, or virtual machine processing may also be constructed to implement the methods described herein.

Although the present disclosure describes components and functions implemented in the embodiments with reference to particular standards and protocols, the disclosure is not limited to such standards and protocols. Other similar standards and protocols not mentioned herein are in existence and are considered to be included in the present disclosure. Moreover, the standards and protocols mentioned herein and other similar standards and protocols not mentioned herein are periodically superseded by faster or more effective equivalents having essentially the same functions. Such replacement standards and protocols having the same functions are considered equivalents included in the present disclosure.

While the foregoing is directed to embodiments of the present disclosure, other and further embodiments of the present disclosure may be devised without departing from the basic scope thereof. It is understood that various embodiments described herein may be utilized in combination with any other embodiment described, without departing from the scope contained herein. Further, the foregoing description is not intended to be exhaustive or to limit the present disclosure to the precise form disclosed. Modifications and variations are possible in light of the above teachings or may be acquired from practice of the present disclosure.

What is claimed is:

1. A method for operating an unmanned aerial vehicle (UAV) under direction from a contact center, the method comprising:
receiving, by a processor of the UAV, dispatch instructions from a computer system of the contact center; wherein:
a) the dispatch instructions comprise a target destination associated with a new contact received by the contact center, the new contact comprising a request from a user; and
b) the UAV is selected by the computer system of the contact center from a plurality of available UAVs for dispatch based on a first set of information received by the contact center related to the contact object and at least one feature of each of the plurality of UAVs;
controlling, by the processor of the UAV, operation of the UAV to navigate the UAV to the target destination;
transmitting, by the processor of the UAV, to the computer system of the contact center, an additional set of information related to the UAV, wherein the additional set of information is transmitted during a travel period of the UAV to the target destination;
receiving, by the processor of the UAV, from the computer system of the contact center, communication instructions; and
in response to receiving the communication instructions, controlling, by the processor of the UAV, an active communication link between the user and a particular agent of the contact center, wherein the particular agent is selected by the computer system of the contact center based on the first set of information and the additional set of information.

2. The method of claim 1, wherein receiving the communication instructions comprises:
receiving, by the processor of the UAV, from the computer system of the contact center, a first optimal time period to establish the active communication link between the user and the particular agent of the contact center.

3. The method of claim 2, wherein controlling the active communication link between the user and the particular agent comprises:
establishing, by the processor of the UAV during the first optimal time period, the active communication link between the user and the particular agent of the contact center.

4. The method of claim 2, further comprising, after transmitting the additional set of information related to the UAV and receiving the optimal time period to establish the active communication link:
receiving, by the processor of the UAV, from the computer system of the contact center, a second optimal time period to establish the active communication link between the user and the particular agent of the contact center, the second optimal time period being different from the first optimal time period; and
establishing, by the processor of the UAV during the second optimal time period, the active communication link between the user and the particular agent of the contact center.

5. The method of claim 1, wherein controlling the active communication link between the user and the particular agent comprises:
activating, by the processor of the UAV, in response to instructions from the computer system of the contact center, at least one of audio or visual communication equipment mounted in or on a body of the UAV.

6. The method of claim 5, wherein the at least one of audio or visual communication equipment comprises at least one of a camera, a light, a video display screen, a speaker, or a microphone.

7. The method of claim 1, wherein the UAV comprises an onboard payload, the method further comprising:
delivering, by the processor of the UAV, in response to instructions from the computer system of the contact center, the onboard payload to the target destination.

8. The method of claim 7, wherein delivering the onboard payload comprises providing the onboard payload to the user.

9. The method of claim 1, further comprising:
transmitting, by the processor of the UAV, to the computer system of the contact center, a further set of information upon arrival at the target destination.

10. The method of claim 9, further comprising:
in response to transmitting the further set of information, receiving, by the processor of the UAV, from the computer system of the contact center, at least one additional instruction.

11. The method of claim 9, wherein the at least one additional instruction comprises one or more instructions to establish a second active communication link between the user and a second agent of the contact center.
